(19) United States
(12) Patent Application Publication
(10) Pub. No.: US 2023/0218185 A1
(43) Pub. Date: Jul. 13, 2023

(54) BIOLOGICAL INFORMATION PROCESSOR, BIOLOGICAL INFORMATION PROCESSING METHOD, AND BIOLOGICAL INFORMATION PROCESSING PROGRAM

(71) Applicant: SONY GROUP CORPORATION, TOKYO (JP)

(72) Inventor: KIMINOBU NISHIMURA, Tokyo (JP)

(21) Appl. No.: 17/998,257

(22) PCT Filed: Apr. 27, 2021
(86) PCT No.: PCT/JP2021/016714

§ 371 Date: Nov. 8, 2022

(30) Foreign Application Priority Data
May 22, 2020 (JP) 2020-089403

(51) Int. Cl. A61B 5/0245; A61B 5/145; A61B 5/11

(52) U.S. Cl. CPC A61B 5/0245; A61B 5/14532; A61B 5/1118

(57) ABSTRACT

A biological information processor including a biological information estimating unit that estimates biological information on a subject in a second period on the basis of a correlation between a first parameter in a first period and biological information in the first period and the first parameter in the second period subsequent to the first period, the first parameter being acquired in advance for the subject.

### TECHNICAL FIELD

The present technique relates to a biological information processor, a biological information processing method, and a biological information processing program.

### BACKGROUND ART

Various biological information sensors have been recently downsized, and devices having the function of measuring biological information, e.g., wearable devices have become widespread in recent years. Accordingly, biological information including heart rates during sports such as running has been measured to manage body conditions and loads of exercise to bodies.

Such a biological information sensor on an arm may fail to perform a correct measurement at low temperatures or when the sensor is in insufficient contact with the arm. A technique is proposed to estimate biological information when the biological information is unmeasurable (PTL 1).

### CITATION LIST

### Patent Literature

### [PTL 1]

### JP 2017-164036 A

### SUMMARY

### Technical Problem

In the technique described in PTL 1, however, characteristics parameters such as VO2Max for a heart rate of each person are not considered, and thus time series variations in heart rate cannot be reflected in the estimation results of heart rates. This may deteriorate an estimation system.

The present technique has been made in view of such a problem, and an object thereof is to provide a biological information processor, a biological information processing method, and a biological information processing program, by which biological information can be estimated with high accuracy when the biological information cannot be correctly measured.

### Solution to Problem

In order to solve the problem, a first technique is a biological information processor including a biological information estimating unit that estimates biological information on a subject in a second period on the basis of a correlation between a first parameter in a first period and biological information in the first period and the first parameter in the second period subsequent to the first period, the first parameter being acquired in advance for the subject.

A second technique is a biological information processing method including estimating biological information on a subject in a second period on the basis of a correlation between a first parameter in a first period and biological information in the first period and the first parameter in the second period subsequent to the first period, the first parameter being acquired in advance for the subject.

A third technique is a program that causes a computer to perform a biological information processing method including estimating biological information on a subject in a second period on the basis of a correlation between a first parameter in a first period and biological information in the first period and the first parameter in the second period subsequent to the first period, the first parameter being acquired in advance for the subject.

### BRIEF DESCRIPTION OF DRAWINGS

FIG. 1 is a block diagram illustrating the configuration of a device 10.

FIG. 2 is a block diagram illustrating another configuration of the device 10.

FIG. 3 is a block diagram illustrating the configuration of a first biological information processor 100.

FIG. 4 is an explanatory drawing of VO2Max.

FIG. 5 is a block diagram illustrating the configuration of a second biological information processor 200.

FIG. 6 is a flowchart of determination.

FIG. 7 is a flowchart indicating the construction of a correlation database.

FIG. 8 is a graph for explaining a time, a heart rate, and an exercise intensity.

FIG. 9 is a flowchart indicating the estimation of biological information.

FIG. 10 is an explanatory drawing of a method of acquiring a convergence heart rate with reference to a correlation database 300.

FIG. 11A is a graph indicating an example of a heart rate estimation result according to the related art, and FIG. 11B is a graph indicating an example of a heart rate estimation result according to the present technique.

FIG. 12 is a block diagram illustrating the configuration of a notification device 400 in a first application example.

FIG. 13 is a block diagram illustrating an example of biological information display in the first application example.

FIG. 14 is a diagram illustrating an example of biological information display in the first application example.

FIG. 15 is a block diagram illustrating the configuration of a determination device 500 in a second application example.

FIG. 16 is a diagram illustrating an example of biological information display in a third application example.

FIG. 17A is a block diagram illustrating the configuration of a difference calculator 600 in a fourth application example, and FIG. 17B is a diagram illustrating an example of biological information display in the fourth application example.

FIG. 18 is a block diagram illustrating the configuration of a second biological information processor 200B in a sixth application example.

FIG. 19 is a diagram indicating an example of a VO2Max database in the sixth application example.

FIG. 20 is a diagram illustrating an example of biological information display in the sixth application example.

FIG. 21 is a block diagram illustrating the configuration of a first biological information processor 100C in a seventh application example.

FIG. 22 is a block diagram illustrating the configuration of a second biological information processor 200C in the seventh application example.

FIG. 23 is a block diagram illustrating the configuration of a lifestyle analyzer 700 in an eighth application example.

FIG. 24 is a diagram indicating that variations in blood sugar level are different because of a difference in insulin secretory ability in a ninth application example.

FIG. 25 is a block diagram illustrating the configuration of a first biological information processor 100D in the ninth application example.

FIG. 26 is a block diagram illustrating the configuration of a second biological information processor 200D in the ninth application example.

FIG. 27 is a graph indicating that variations in blood alcohol concentration are different because of a difference in alcohol metabolizing ability in a tenth application example.

FIG. 28 is an explanatory drawing of a parameter and biological information in an eleventh application example.

FIG. 29 is a diagram indicating a specific example of behaviors/conditions and biological characteristic parameters in the eleventh application example.

### DESCRIPTION OF EMBODIMENTS

Hereinafter, an embodiment of the present technique will be described with reference to the drawings. Hereinafter descriptions will proceed in the following order.

### <1. Embodiment>

[1-1. Configuration of device 10]
[1-2. Configuration of first biological information processor 100]
[1-3. Configuration of second biological information processor 200]

### [1-4. Determination]

[1-5. Correlation database construction]
[1-6. Heart rate estimation]
<2. Application example>
<3. Modification example>

### 1. Embodiment

### [1-1. Configuration of Device 10]

The processing of the present technique includes two steps: database construction for estimating biological information, and biological information estimation using a database. A database is constructed by a first biological information processor 100, and biological information is estimated by a second biological information processor 200. In this embodiment, biological information is a heart rate of a user serving as a subject. First, the configuration of a device 10, in which the first biological information processor 100 and the second biological information processor 200 operate, will be described below.

The device 10 includes a control unit 11, a storage unit 12, an interface 13, an input unit 14, a display unit 15, a heart rate sensor 16, an acceleration sensor 17, the first biological information processor 100, and the second biological information processor 200.

The control unit 11 includes a CPU (Central Processing Unit), a RAM (Random Access Memory), and a ROM (Read Only Memory). The CPU controls the overall device 10 and each unit thereof by performing various types of processing according to programs stored in the ROM and issuing commands.

The storage unit 12 is, for example, a large-capacity storage medium such as a hard disk or a flash memory. In the storage unit 12, for example, various applications running in the device 10 are stored and data used in the first biological information processor 100 and the second biological information processor 200 is stored.

The interface 13 is an interface between the Internet and other devices. The interface 13 may include a wire or radio communication interface. More specifically, the wire or radio communication interface may include cellular communications such as 3TTE, Wi-Fi, Bluetooth (registered trademark), NFC (Near Field Communication), Ethernet (registered trademark), HDMI (registered trademark), (High-Definition Multimedia Interface), and USB (Universal Serial Bus). If at least some of the device 10, the first biological information processor 100, and the second biological information processor 200 are implemented in the same unit, the interface 13 may include a bus in the unit and data reference in a program module (hereinafter also referred to as an interface in the unit). If the device 10 and the biological information processors are dispersed in multiple units, the interface 13 may include a variety of interfaces for the respective units. For example, the interface 13 may include a communication interface and an interface in a unit.

The input unit 14 allows a user to input various instructions to the device 10. In response to a user input to the input unit 14, a control signal corresponding to the input is generated and is supplied to the control unit 11. The control unit 11 then performs various types of processing in response to the control signal. The input unit 14 includes a touch panel, a voice input by voice recognition, and a gesture input by body recognition in addition to physical buttons.

The display unit 15 is, for example, a display that displays a GUI (Graphical User Interface) for displaying image/video, database construction, biological information estimation, and estimated biological information.

The heart rate sensor 16 is a known heart rate sensor that measures a heart rate of the user serving as a subject. The heart rate sensor 16 can measure a heart rate of the user when the user takes a rest or exercises. The heart rate can be expressed in BPM (Beat Per Minute) units.

The acceleration sensor 17 is a sensor capable of measuring an acceleration in, for example, a known biaxial or triaxial direction. The acceleration sensor 17 can measure the acceleration of a user body part, to which the device 10 is attached, while the user exercises.

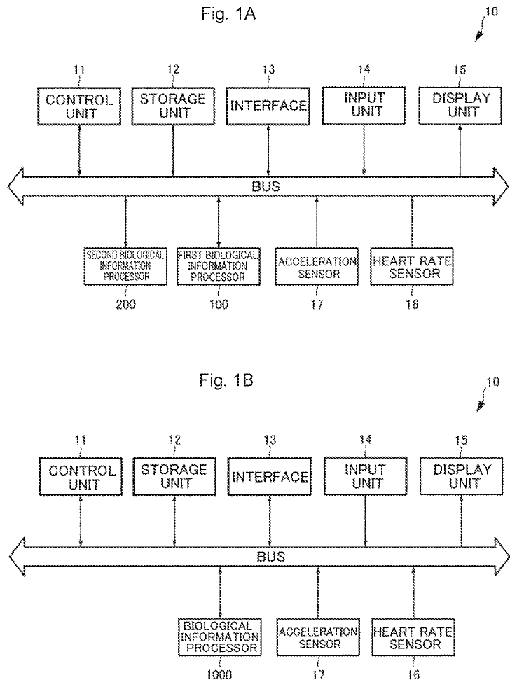

The first biological information processor 100 and the second biological information processor 200 may operate in the same device 10 as illustrated in FIG. 1A. Alternatively, a biological information processor 1000 having the functions of the first biological information processor 100 and the second biological information processor 200 may operate in the device 10 as illustrated in FIG. 1B.

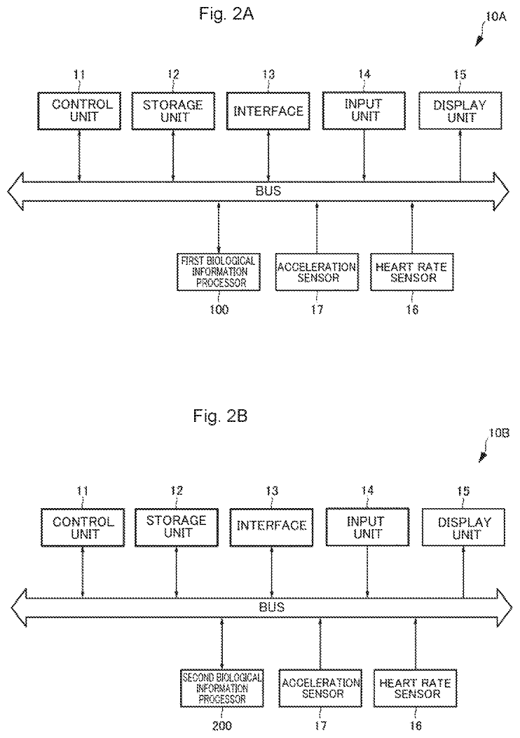

As illustrated in FIGS. 2A and 2B, the first biological information processor 100 and the second biological information processor 200 may operate in different devices 10A and 10B. The heart rate sensor 16 and the acceleration sensor 17 may be configured separately from the device 10 and may transmit sensor information to the device 10 through a network or Bluetooth (registered trademark) communications.

The device 10 is, for example, a wearable device, a smartphone, a tablet, or a personal computer. If the device 10 includes the heart rate sensor 16 and the acceleration sensor 17, the device 10 needs to be wearable by the user. If the device 10 includes at least the acceleration sensor 17 (the heart rate sensor 16 is configured as a separate device), the device 10 needs to be portable by a user like a smartphone or a wearable device.

### [1-2. Configuration of First Biological Information Processor 100]

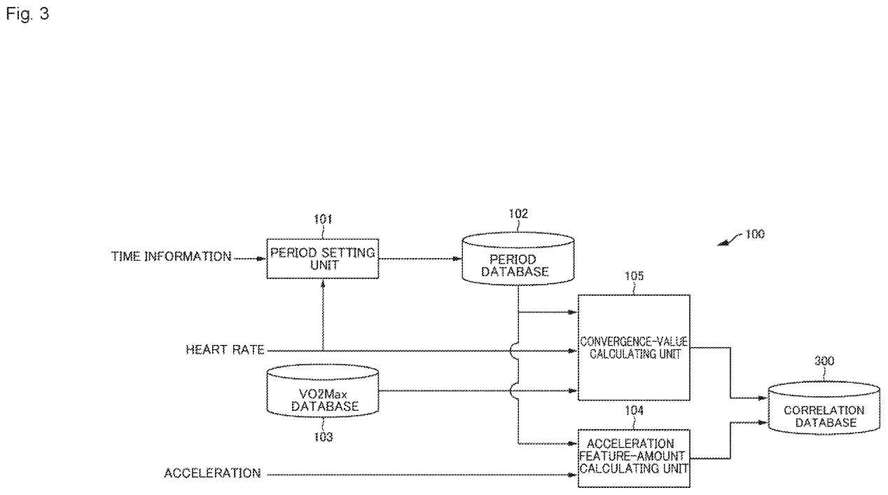

Referring to FIG. 3, the configuration of the first biological information processor 100 will be described below. The first biological information processor 100 constructs a correlation database 300 used for estimating a heart rate as biological information. The first biological information processor 100 includes a period setting unit 101, a period database 102, a VO2Max database 103, an acceleration feature-amount calculating unit 104, a convergence-value calculating unit 105, and the correlation database 300.

The period setting unit 101 sets a first period from a time period during which the user continues an exercise. The first period is a period for which the convergence-value calculating unit 105 calculates a convergence heart rate HR^{(x)}ₚₗₐₜ and a period for which the acceleration feature-amount calculating unit 104 calculates an acceleration feature amount AC_{feat}. The period setting unit 101 receives, for example, time information from a clock function ordinarily provided for the device 10.

Moreover, the period setting unit 101 receives a hear rate of the user, the heart rate being measured by the heart rate sensor 16. For example, as a starting time tₛₜₐᵣₜ of the first period, the period setting unit 101 sets a time after the lapse of the predetermined time from the start of measurement of a heart rate by the heart rate sensor 16. As a finishing time t_{end} of the first period, the period setting unit 101 sets a time after the lapse of a predetermined time from the start of the first period.

The period database 102 is provided for storing data on the starting time tₛₜₐᵣₜ and the finishing time t_{end} of the first period, the starting and finishing times being set by the period setting unit 101. The period database 102 may be configured in the storage unit 12 of the device 10.

The VO2Max database 103 is provided for storing VO2Max that is a user-specific parameter. VO2Max[ml/min/kg] is an index indicating cardiorespiratory endurance. Generally, elderly people and people who do not exercise in their daily life tend to have a low VO2Max value. A user of the first biological information processor 100 needs to acquire VO2Max in advance for the user and store the VO2Max in the VO2Max database 103. The VO2Max corresponds to a second parameter in the claims. The VO2Max database 103 may be configured in the storage unit 12 of the device 10.

In the present technique, VO2Max is used to estimate a heart rate because VO2Max varies among persons and heart rate characteristics vary with VO2Max.

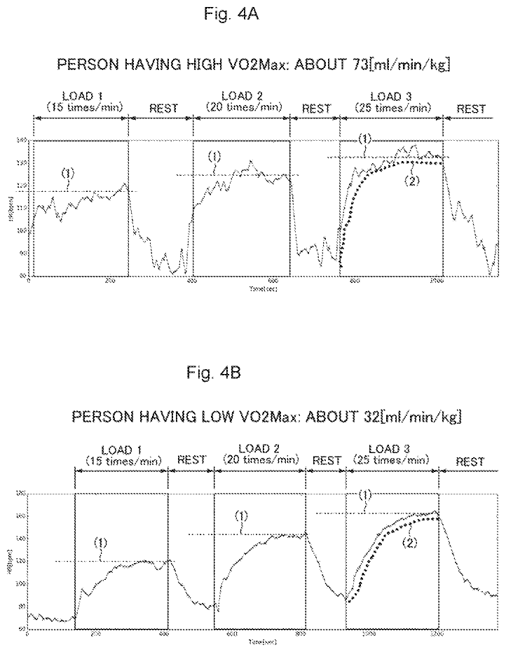

As indicated in FIG. 4, when the same load is applied (e.g., during an exercise) to a person having VO2Max of about 73[ml/min/kg] (high VO2Max) and a person having VO2Max of about 32[ml/min/kg] (low VO2Max), the characteristics of rising heart rates are different from each other. As indicated by (1) in FIG. 4, the person having low VO2Max tends to have a higher heart rate than the person having high VO2Max during the same exercise. As indicated by (2) in FIG. 4, the person having high VO2Max typically rises in heart rate earlier than the person having low VO2Max, and the person having low VO2Max more gradually rises in heart rate than the person having high VO2Max. Thus, a heart rate can be estimated with higher accuracy by using VO2Max.

The acceleration feature-amount calculating unit 104 calculates the acceleration feature amount AC_{feat} in the first period. The acceleration feature amount AC_{feat} is a value calculated from the acceleration of an exercise of the user in the first period, the acceleration being measured by the acceleration sensor 17. The acceleration feature amount AC_{feat} corresponds to a first parameter in the claims.

The convergence-value calculating unit 105 calculates a convergence heart rate HR^{(x)}ₚₗₐₜ that is a heart rate converging when the user continues an exercise with an exercise intensity x in the first period. The convergence-value calculating unit 105 receives the starting time tₛₜₐᵣₜ and the finishing time t_{end} of the first period from the period database 102, the heart rate of the user from the heart rate sensor 16, and VO2Max from the VO2Max database 103. The exercise intensity is a value that can be defined in units of km/h. The detail will be described later.

The correlation database 300 is provided for storing the acceleration feature amount AC_{feat} and the convergence heart rate HR^{(x)}ₚₗₐₜ, which are calculated for the first period, such that the feature amount and the heart rate are associated with each other. The acceleration feature amount AC_{feat} and the convergence heart rate HR^{(x)}ₚₗₐₜ, which are stored in the correlation database 300, are used for estimating a heart rate by the second biological information processor 200. Data stored in the correlation database 300 corresponds to a correlation between the first parameter and biological information in the claims. The correlation database 300 may be configured in the storage unit 12 of the device 10.

### [1-3. Configuration of Second Biological Information Processor 200]

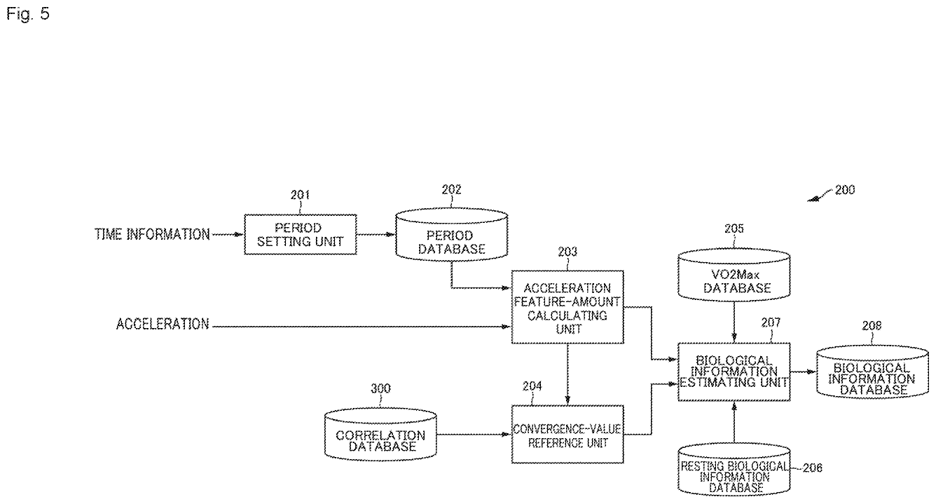

Referring to FIG. 5, the configuration of the second biological information processor 200 will be described below. The second biological information processor 200 estimates a heart rate of the user as biological information. The second biological information processor 200 includes a period setting unit 201, a period database 202, an acceleration feature-amount calculating unit 203, a convergence-value reference unit 204, a VO2Max database 205, a resting biological information database 206, a biological information estimating unit 207, a biological information database 208, and a correlation database 300.

The period setting unit 201 sets a second period from a time period during which the user continues an exercise. The second period is a period for estimating a heart rate of the user. The period setting unit 101 receives, for example, time information from the clock function ordinarily provided for the device 10.

For example, as a starting time tₛₜₐᵣₜ of the second period, the period setting unit 201 sets a time after the lapse of a predetermined time from the start of measurement of an acceleration by the acceleration sensor 17. As a finishing time t_{end} of the second period, the period setting unit 201 sets a time after the lapse of a predetermined time from the start of the second period.

The period database 202 is provided for storing data on the starting time tₛₜₐᵣₜ and the finishing time t_{end} of the second period, the starting and finishing times being set by the period setting unit 201. The period database 202 may be configured in the storage unit 12 of the device 10.

The acceleration feature-amount calculating unit 203 calculates the acceleration feature amount AC_{feat} in the second period. The acceleration feature amount AC_{feat} is a value calculated from the acceleration of an exercise of the user in the second period, the acceleration being measured by the acceleration sensor 17. The acceleration feature amount AC_{feat} corresponds to the first parameter in the claims.

Referring to the correlation database 300 by using the calculated acceleration feature amount AC_{feat}, the convergence-value reference unit 204 acquires the convergence heart rate HR^{(x)}ₚₗₐₜ corresponding to the acceleration feature amount AC_{feat}.

The VO2Max database 205 is provided for storing VO2Max that is a user-specific parameter. The VO2Max database 205 is identical to that of the first biological information processor 100. The VO2Max database 205 may be configured in the storage unit 12 of the device 10.

The resting biological information database 206 is provided for storing a resting heart rate HRᵢₙᵢₜ of the user. The resting heart rate HRᵢₙᵢₜ is a heart rate when the acceleration of a user action is 0 or not higher than a predetermined value, that is, a resting heart rate. If the resting heart rate HRᵢₙᵢₜ for health care is acquired in advance by the user, the resting heart rate HRᵢₙᵢₜ can be used regardless of the use of the second biological information processor 200. If the resting heart rate HRᵢₙᵢₜ is not acquired in advance, the user needs to acquire a resting heart rate by measurement using the heart rate sensor 16 of the device 10 in which the biological information processor operates, or another heart rate sensor 16. The resting biological information database 206 may be configured in the storage unit 12 of the device 10.

The biological information estimating unit 207 estimates a heart rate HR(t) of the user at any time t in the second period by calculation on the basis of the acceleration feature amount AC_{feat}, VO2Max, the convergence heart rate HR^{(x)}ₚₗₐₜ, and the resting heart rate HRᵢₙᵢₜ.

The biological information database 208 is provided for storing a heart rate HR(t) of the user at any time, the heart rate being calculated by the biological information estimating unit 207. The biological information database 208 may be configured in the storage unit 12 of the device 10.

The correlation database 300 is provided for storing the convergence heart rate HR^{(x)}ₚₗₐₜ and the acceleration feature amount AC_{feat}, which are calculated for the first period by the first biological information processor 100, such that the heart rate and the feature amount are associated with each other. The correlation database 300 is identical to that of the first biological information processor 100.

If the second biological information processor 200 operates in the same device as the first biological information processor 100, the period setting unit 201, the period database 202, the acceleration feature-amount calculating unit 203, the VO2Max database 205, and the correlation database 300 may be shared with the first biological information processor 100. If the first biological information processor 100 and the second biological information processor 200 operate in different devices, the correlation database 300 constructed by the first biological information processor 100 needs to be shared by the second biological information processor 200 via, for example, a network or direct communications.

The first biological information processor 100 and the second biological information processor 200 are configured as follows: The first biological information processor 100 and the second biological information processor 200 may be configured as separate devices or may operate in the device 10. A control program for processing according to the present technique may be installed in the device 10 in advance or may be installed by a provider after being downloaded or distributed by a storage medium or the like.

### [1-4. Determination]

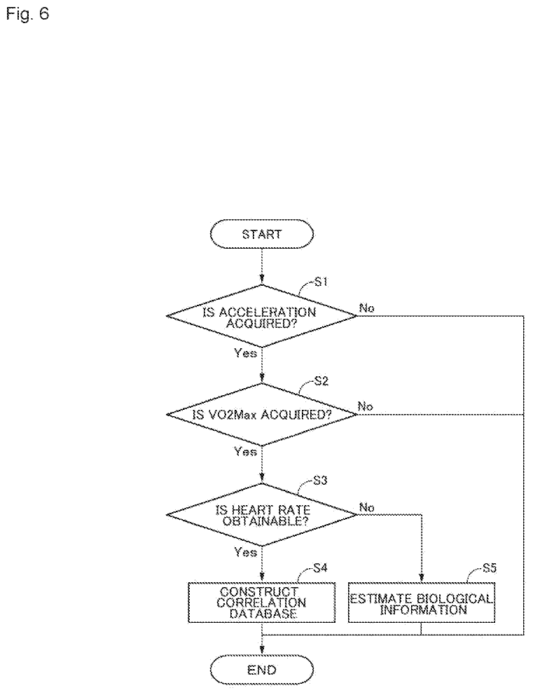

Referring to the flowchart of FIG. 6, processing for determining whether to perform correlation database construction by the first biological information processor 100 or biological information estimation by the second biological information processor 200 will be described below. This processing is performed by the device 10 to determine whether to perform the processing of the first biological information processor 100 or the second biological information processor 200 in the case where the first biological information processor 100 and the second biological information processor 200 operate in the single device 10 as illustrated in FIGS. 1A and 1B.

First, in step S1, whether the acceleration of a user action can be acquired from the acceleration sensor 17 is confirmed. If the acceleration can be acquired, the processing advances to step S2 (Yes at step S1). If the acceleration cannot be acquired, the correlation database 300 cannot be constructed or biological information cannot be estimated, so that the processing is terminated (No at step S1).

In step S2, whether the VO2Max of the user has been acquired is confirmed. If VO2Max has been acquired, the processing advances to step S3 (Yes at step S2). If VO2Max has not been acquired, the correlation database 300 cannot be constructed or biological information cannot be estimated, so that the processing is terminated (No at step S2).

In step S3, whether the heart rate of the user can be acquired from the heart rate sensor 16 is confirmed. If the heart rate can be acquired, the correlation database is constructed by the first biological information processor 100 in step S4 (Yes at step S3).

If the heart rate cannot be acquired, the heart rate is estimated by the second biological information processor 200 in step S5 (No at step S3). The heart rate cannot be acquired in the following case: the user wearing the heart rate sensor 16 is actively exercising and thus the heart rate cannot be correctly measured or a heart rate can be measured in a limited way, or a heart rate cannot be measured because the user does not wear the heart rate sensor 16.

If the first biological information processor 100 and the second biological information processor 200 operate in different devices 10A and 10B as illustrated in FIG. 2, the user needs to confirm the same processing contents and determine whether to perform correlation database construction or biological information estimation. The user uses one of the device 10A and the device 10B according to processing to be performed.

### [1-5. Correlation Database Construction]

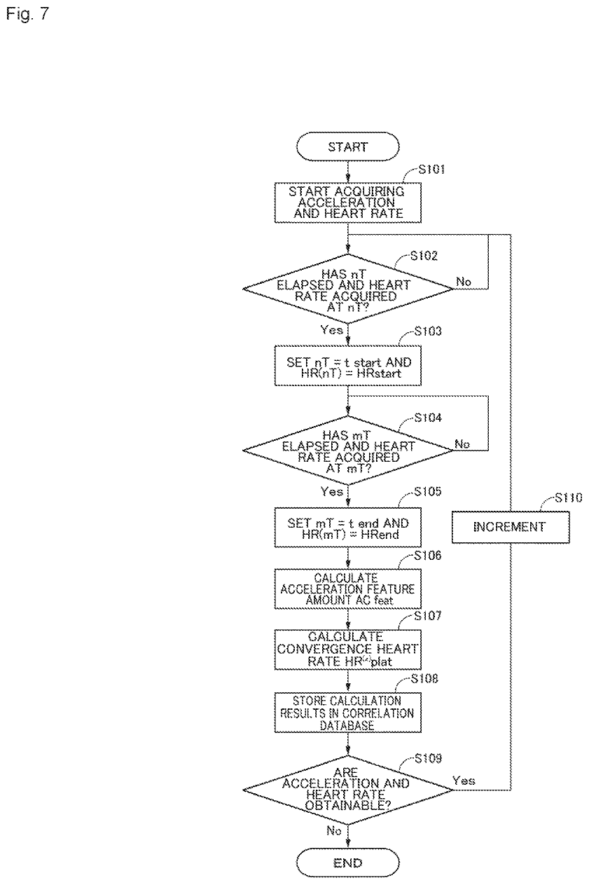

Referring to the flowchart of FIG. 7, processing performed by the first biological information processor 100 will be described below. This processing is performed to construct the correlation database 300 used for estimating a heart rate by the second biological information processor 200.

In order to perform the processing, the user needs to wear the device 10 including the heart rate sensor 16, the acceleration sensor 17, and the first biological information processor 100 or wear the heart rate sensor 16 and the acceleration sensor 17 that are separate devices. If the heart rate sensor 16 and the acceleration sensor 17 are separate devices, the first biological information processor 100 needs to acquire a heart rate and an acceleration from the heart rate sensor 16 and the acceleration sensor 17 via a network or Bluetooth (registered trademark) communications.

When the heart rate sensor 16 starts measuring the heart rate of the user and the acceleration sensor 17 starts measuring the acceleration of a user action, the first biological information processor 100 first starts acquiring a heart rate and acceleration information in step S101.

Subsequently, in step S102, the period setting unit 101 confirms whether nT seconds (n=1, 2, 3, . . . N) have elapsed from the start of acquisition of a heart rate and an acceleration and a heart rate has been acquired at nT seconds after the start of acquisition of an acceleration. Since the initial value of n is 1, the period setting unit 101 in the first cycle of processing first confirms whether T seconds have elapsed from the start of acquisition of a heart rate and an acceleration and a heart rate has been acquired at T seconds after the start of acquisition of an acceleration.

If a heart rate has not been acquired at nT seconds, step S102 is repeated until a heart rate is acquired (No at step S102). If nT seconds have elapsed and a heart rate HR(nT) has been acquired at nT seconds after the start of measurement of an acceleration, the processing advances to step S103 (Yes at step S102).

In step S103, the period setting unit 101 sets nT at the starting time tₛₜₐᵣₜ of the first period (tₛₜₐᵣₜ=nT) and sets HR(nT), which is a heart rate at nT, at HRₛₜₐᵣₜ that is a heart rate of the starting time tₛₜₐᵣₜ of the first period (HRₛₜₐᵣₜ=HR(nT)).

In step S104, the period setting unit 101 confirms whether mT seconds (m=n+1, n+2, n+3, n+N) have elapsed from the starting time tₛₜₐᵣₜ of the first period and a heart rate has been acquired at mT seconds after the starting time tₛₜₐᵣₜ of the first period. Since the initial value of m is n+1, the period setting unit 101 in the first cycle of processing first confirms whether (n+1)T seconds have elapsed from the start of acquisition of a heart rate and an acceleration and a heart rate has been acquired at (n+1)T seconds after the start of acquisition of an acceleration.

If a heart rate has not been acquired at mT seconds, step S104 is repeated until a heart rate is acquired (No at step S104). If mT seconds have elapsed and a heart rate HR(mT) has been acquired at mT seconds after the starting time tₛₜₐᵣₜ of the first period, the processing advances to step S105 (Yes at step S104).

In step S105, the period setting unit 101 sets mT at the finishing time t_{end} of the first period (t_{end}=mT) and sets HR(mT), which is a heart rate at mT, at HR_{end} that is a heart rate at the end of the first period (HR_{end}=HR(mT)).

In step S106, the acceleration feature-amount calculating unit 104 calculates the acceleration feature amount AC_{feat} in the first period from tₛₜₐᵣₜ to t_{end} according to expression 1 below. As indicated by expression 1, the acceleration feature amount AC_{feat} can be calculated as an acceleration norm from which a gravity component has been subtracted.

∥norm[G]−1[G]∥ [Math. 1]

Additionally, the acceleration feature amount AC_{feat} may be the number of steps, the acceleration norm of a gravitational directional axis, or the integral of the acceleration norm of the gravitational directional axis in a certain time.

The acceleration feature amount AC_{feat} includes, for example, a qualitative element that is a body part of the user wearing the acceleration sensor 17, for example, “the right wrist: AC norm average 2.3 G”, and a quantitative element that is an acceleration value calculated as has been discussed.

In step S107, according to expression 2 below, the convergence-value calculating unit 105 calculates the convergence heart rate HR^{(x)}ₚₗₐₜ that is a heart rate converging when the user continues an exercise with the exercise intensity x in the first period from tₛₜₐᵣₜ to t_{end}. τ is a time from tₛₜₐᵣₜ to t_{end} of the first period.

$${HR}_{plat .}^{( x )} = \frac{HR ( τ ) - {HR}_{init .} {e}^{- ατ}}{1 - {e}^{- ατ}} \qquad [ Math . 2 ]$$

Step S106 and step S107 may be simultaneously performed in parallel.

In step S108, the acceleration feature amount AC_{feat} and the convergence heart rate HR^{(x)}ₚₗₐₜ are stored in the correlation database 300 so as to be associated with each other.

In step S109, it is confirmed whether a heart rate can be acquired from the heart rate sensor 16 and whether an acceleration can be acquired from the acceleration sensor 17. If the heart rate and the acceleration can be acquired, the processing advances to step S110 to increment n (Yes at step S109). If a heart rate and an acceleration can be acquired, steps from S102 to 110 are repeated to construct the correlation database 300 in which the acceleration feature amount AC_{feat} and the convergence heart rate HR^{(x)}ₚₗₐₜ are associated with each other in a period during which the heart rate and the acceleration can be acquired.

If one or both of a heart rate and an acceleration cannot be acquired, the processing is terminated (No at step S109). For example, if the user stops exercising and the acceleration decreases to 0 or a predetermined value or lower, the acceleration of the user cannot be acquired.

The correlation database 300 in which the acceleration feature amount AC_{feat} and the convergence heart rate HR^{(x)}ₚₗₐₜ are associated with each other can be constructed thus.

A method of calculating the convergence heart rate HR^{(x)}ₚₗₐₜ by the convergence-value calculating unit 105 will be described below. If the user starts an exercise from a rest, a heart rate HR(t) at t seconds after the start of the exercise can be physiologically estimated in consideration of heart rate characteristics according to expression 3 below.

HR(t)=HR_{plat.}^{(x)}(1−e^{-αt})+HR_{init.}e^{-αt} [Math. 3]

where x is an exercise intensity. If the resting heart rate HRᵢₙᵢₜ is known, the heart rate HRτ obtained after the lapse of the time τ from the start of the exercise is known, and α that is a value obtained by multiplying VO2Max by a coefficient is known, the exercise intensity x can be calculated from the values.

The coefficient for the multiplication of VO2Max to calculate a is determined on the basis of the relationship between VO2Max and an increase in heart rate when a constant exercise intensity is continued. A coefficient determined for another user can be also used. The coefficient is desirably determined for each person in order to increase the accuracy of estimation of a heart rate.

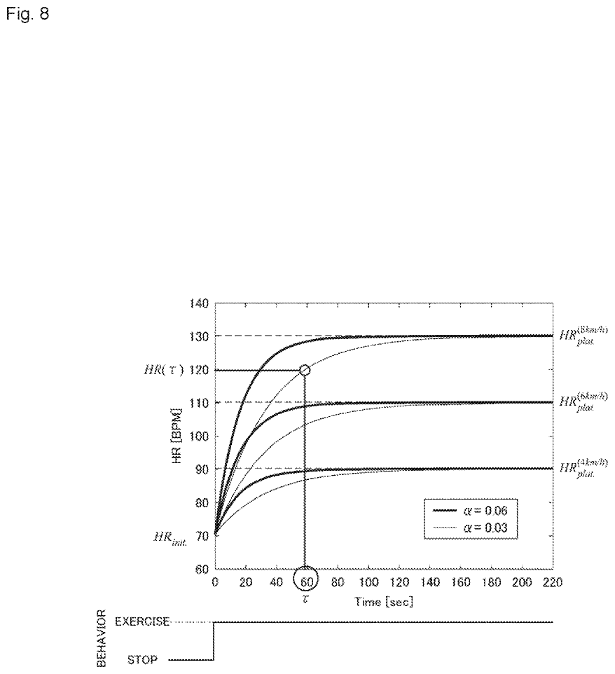

FIG. 8 is a graph of an elapsed time from the start of an exercise and a heart rate when a person having high VO2Max (thick lines) and a person having low VO2Max (thin lines) do the same exercise in three patterns (running at 4 km/h, 6 km/h, 8 km/h).

For example, in the case of the person having low VO2Max in the graph of FIG. 8, if a resting heart rate HRᵢₙᵢₜ, which is a heart rate at the start of an exercise, is 70, a time τ is 60 seconds from the start of the exercise, a heart rate HRτ is 120 after the lapse of the time τ (60 seconds) from the start of the exercise, and a is 0.03, the exercise intensity x can be estimated to be 8 km/h.

Furthermore, the foregoing expression 1 can be derived by solving expression 3 with respect to the convergence heart rate HR^{(x)}ₚₗₐₜ. By using expression 1, the convergence heart rate HR^{(x)}ₚₗₐₜ can be calculated in the case of the exercise intensity x is continued for the time τ from tₛₜₐᵣₜ to t_{end}.

The convergence heart rate HR^{(x)}ₚₗₐₜ can be expressed by an exercise intensity instead of a heart rate obtained when the exercise intensity x is continued. The exercise intensity can be expressed by % VO2Max according to the Karvonen method.

The correlation database 300 in which the acceleration feature amount AC_{feat} and the convergence heart rate HR^{(x)}ₚₗₐₜ are associated with each other can be constructed thus. In order to accurately estimate a heart rate according to various exercises by the user, various situations, and the conditions of the user, it is preferable to maximize the acceleration feature amount AC_{feat} and the convergence heart rate HR^{(x)}ₚₗₐₜ that are stored in the correlation database 300.

If a heart rate is to be estimated in a specific situation, e.g., a sports game, the correlation database 300 may be constructed by using the first biological information processor 100 in a situation close to the sports game, for example, in the practice of the sport. Since a heart rate in a game is estimated on the basis of data in the practice of the same sport, the estimation can be more accurate than estimation based on data about completely different sports or exercises.

### [1-6. Heart Rate Estimation]

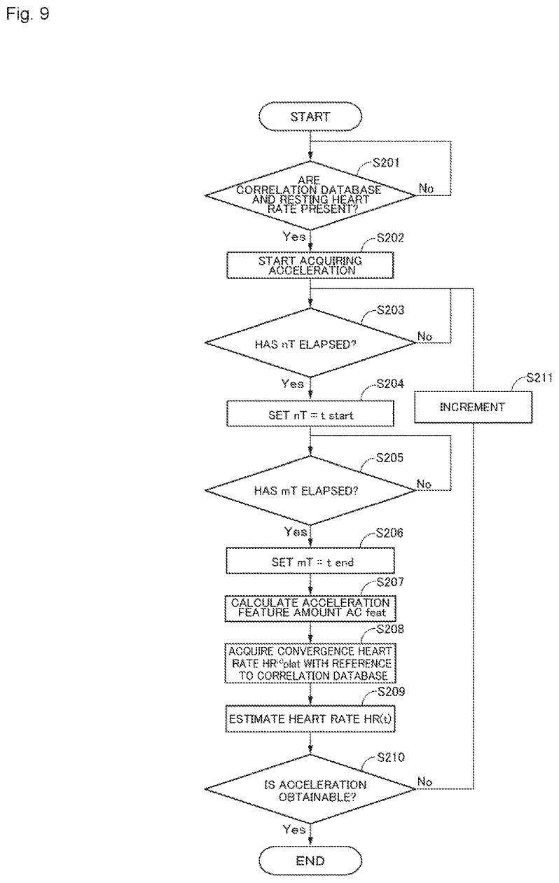

Referring to the flowchart of FIG. 9, the estimation of a heart rate by the second biological information processor 200 will be described below. The estimation of a heart rate is processing for estimating a heart rate of the user in the second period subsequent to the first period. The second period corresponds to any one of the following cases: the case where the user wearing the heart rate sensor 16 is actively exercising and thus a heart rate cannot be correctly acquired, the case where a heart rate can be acquired only in a limited way, and the case where a heart rate cannot be acquired because the user does not wear the heart rate sensor 16. The acquisition in a limited way indicates that a heart rate can be acquired in a limited period or a heart rate cannot be acquired in some periods such that some of the heart rate measurement results are missing.

In order to perform the processing, the user needs to wear the device 10 including the acceleration sensor 17 and the second biological information processor 200 or wear the acceleration sensor 17 that is a separate device. If the acceleration sensor 17 is a separate device, the second biological information processor 200 needs to acquire an acceleration from the acceleration sensor 17 via a network or Bluetooth (registered trademark) communications.

First, in step S201, the second biological information processor 200 confirms the presence or absence of the acceleration feature amount AC_{feat} and the convergence heart rate HR^{(x)}ₚₗₐₜ in the correlation database 300 and the presence or absence of the resting heart rate HRᵢₙᵢₜ in the resting biological information database 206.

When the acceleration sensor 17 starts measuring the acceleration of a user action, the second biological information processor 200 first starts acquiring an acceleration from the acceleration sensor 17 in step S202.

Subsequently, in step S203, the period setting unit 201 confirms whether nT seconds (n=1, 2, 3, . . . N) have elapsed from the start of measurement of an acceleration. Since the initial value of n is 1, the period setting unit 201 in the first cycle of processing first confirms whether T seconds have elapsed from the start of measurement of an acceleration.

If nT seconds have not elapsed, step S203 is repeated until the elapse of nT seconds (No at step S203). If nT seconds have elapsed after the start of measurement of an acceleration, the processing advances to step S204 (Yes at step S203).

In step S204, the period setting unit 201 sets nT at the starting time tₛₜₐᵣₜ of the second period (tₛₜₐᵣₜ=nT).

Subsequently, in step S205, the period setting unit 201 confirms whether mT seconds (m=n+1, n+2, n+3, n+N) have elapsed from the starting time tₛₜₐᵣₜ of the second period. Since the initial value of n is 1, the period setting unit 201 in the first cycle of processing first confirms whether 2T seconds have elapsed from the start of measurement of an acceleration.

If mT seconds have not elapsed, step S205 is repeated until the elapse of mT seconds (No at step S205). If mT seconds have elapsed after tₛₜₐᵣₜ, the processing advances to step S206 (Yes at step S205).

In step S206, the period setting unit 201 sets mT at the finishing time t_{end} of the second period (t_{end}=mT).

In step S207, the acceleration feature-amount calculating unit 203 calculates the acceleration feature amount AC_{feat} in the second period. The method of calculating the acceleration feature amount AC_{feat} is identical to that of the first biological information processor 100.

In step S208, with reference to the correlation database 300, the convergence-value reference unit 204 acquires the convergence heart rate HR^{(x)}ₚₗₐₜ corresponding to the acceleration feature amount AC_{feat}.

In step S209, the biological information estimating unit 207 calculates a heart rate HR(t) at any time t in the second period by using the foregoing expression 3 on the basis of the acceleration feature amount AC_{feat}, VO2Max, the convergence heart rate HR^{(x)}ₚₗₐₜ, and the resting heart rate HRᵢₙᵢₜ. The heart rate HR(t) serves as an estimated heart rate.

In step S210, it is confirmed whether an acceleration can be acquired from the acceleration sensor 17. If the acceleration can be acquired, the processing advances to step S211 to increment n (Yes at step S210). Thereafter, steps S203 to S211 are repeated. Thus, the convergence heart rate HR^{(x)}ₚₗₐₜ from tₛₜₐᵣₜ to tend can be acquired until the acceleration sensor 17 becomes unable to acquire the acceleration of a user action. In the second period, the starting time tₛₜₐᵣₜ is nT and the fining time t_{end} is mT. Thus, the second period is extended as long as the acceleration sensor 17 can acquires an acceleration, so that the convergence heart rate HR^{(x)}ₚₗₐₜ of the second period is calculated.

When the acceleration sensor 17 becomes unable to acquire the acceleration of a user action, the processing is terminated (No at step S210).

A method of acquiring the convergence heart rate HR^{(x)}ₚₗₐₜ will be described below. In this method, with reference to the correlation database 300 by the convergence-value reference unit 204 in step S208, the convergence heart rate HR^{(x)}ₚₗₐₜ corresponding to the acceleration feature amount AC_{feat} is not present.

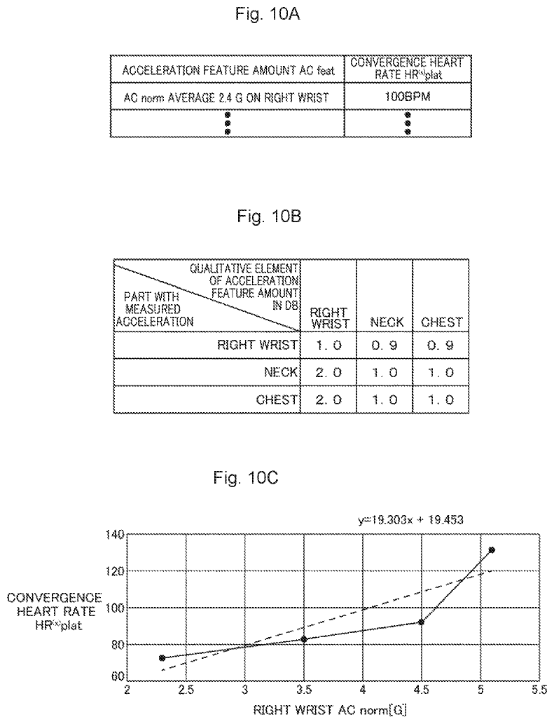

For example, as indicated in FIG. 10A, it is assumed that data stored in the correlation database 300 includes “AC norm average 2.4 G on a right wrist” that is an acceleration feature amount AC_{feat} and “100 BPM” that is a convergence heart rate HR^{(x)}ₚₗₐₜ corresponding to the feature amount. As described above, the acceleration feature amount AC_{feat} includes a qualitative element that is a part having the acceleration sensor 17 and a quantitative element that is an acceleration value.

The data indicated in FIG. 10A is stored in the correlation database 300. It is assumed that the acceleration feature amount AC_{feat} calculated in step S207 is “AC norm average 1.2 G on the neck”. In this case, the qualitative element of the AC_{feat} varies between “right wrist” and “neck”, so that the convergence heart rate HR^{(x)}ₚₗₐₜ cannot be acquired.

Thus, in this case, the quantitative element of the acceleration feature amount AC_{feat} is multiplied by a coefficient to convert the acceleration feature amount AC_{feat}. The coefficient is acquired from, for example, the relational table of coefficients and qualitative elements. The relational table is prepared in advance as indicated in FIG. 10B. In the relational table indicated in FIG. 10B, the coefficients are set for combinations of parts having the acceleration sensor 17 for measuring an acceleration and the qualitative elements of the acceleration feature amount AC_{feat} in the correlation database 300. The coefficients in the relational table can be calculated from the average of the ratios of the acceleration feature amount ACfeat norm after an acceleration during an exercise is measured with the acceleration sensor 17 attached to parts constituting the combinations, e.g., a right wrist and a neck or a right wrist and a chest in the table.

In this example, a qualitative element in the correlation database 300 is “right wrist” and the qualitative element of the acceleration feature amount AC_{feat} calculated in step S207 is “neck”, so that the coefficient can be derived as 2.0.

By multiplying an acceleration, which is the quantitative element of “AC norm average 1.2 G on the neck” serving as the acceleration feature amount AC_{feat}, by the coefficient 2.0, the acceleration feature amount AC_{feat} calculated in step S207 can be converted into “AC norm average 2.4 G on the right wrist”. Thus, referring to the correlation database 300 by using the acceleration feature amount AC_{feat} calculated in step S207, the convergence heart rate HR^{(x)}ₚₗₐₜ “100 BPM” can be acquired.

A method of acquiring the convergence heart rate HR^{(x)}ₚₗₐₜ will be described below. In this method, the data of the correlation database 300 and the quantitative element of the acceleration feature amount AC_{feat} calculated in step S207 are different from each other, so that the convergence heart rate HR^{(x)}ₚₗₐₜ cannot be acquired from the correlation database 300.

For example, if the acceleration feature amount AC_{feat} calculated in step S207 is “AC norm average 2.8 G on the right wrist”, the acceleration feature amount AC_{feat} of the correlation database 300 in FIG. 10A has a quantitative element of 2.4 that is different from 2.8, so that the convergence heart rate HR^{(x)}ₚₗₐₜ cannot be acquired.

In order to acquire the convergence heart rate HR^{(x)}ₚₗₐₜ in this situation, the correlation database 300 is constructed in advance by a plurality of quantitative elements of the acceleration feature amount AC_{feat}. The convergence heart rate HR^{(x)}ₚₗₐₜ is then estimated on the basis of a regression equation. For example, if convergence heart rates HRplat are set as a database in advance for respective four quantitative elements as indicated in the graph of FIG. 10C, regression equations (not limited to linear regression) are obtained for the convergence heart rates, so that a convergence heart rate HR^{(x)}ₚₗₐₜ can be acquired for a quantitative element 2.8 that is not present in the correlation database 300.

With reference to the correlation database 300 by the convergence-value reference unit 204, if the convergence heart rate HR^{(x)}ₚₗₐₜ corresponding to the acceleration feature amount AC_{feat} is not present, the convergence-value reference unit 204 may refer to another correlation database. The convergence-value reference unit 204 desirably refers to a correlation database with VO2Max close to that of the user.

For example, the convergence heart rate can be acquired by connecting the second biological information processor 200 to a network via the device 10 and accessing a cloud, a server, or another device to refer to another VO2Max database and another correlation database.

As described above, a heart rate is estimated as biological information according to the present technique. The present technique can estimate a heart rate with high accuracy on the basis of an acceleration if the relationship between an acceleration and a heart rate is learned in advance, the acceleration can be measured, and the heart rate is unmeasurable, is incorrectly measured, or is measured only in a limited way.

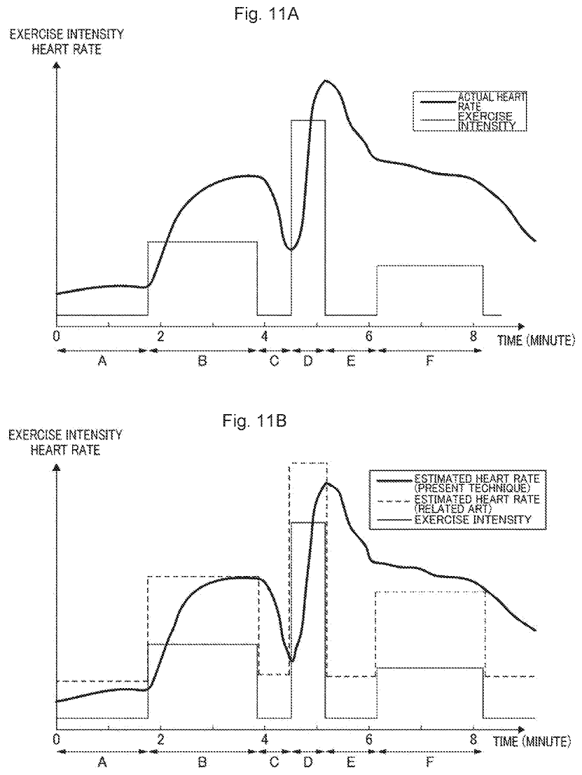

Referring to FIG. 11, an example of the result of estimating a heart rate according to the related art and the result of estimating a heart rate according to the present technique will be described below. In the graphs of FIGS. 11A and 11B, the horizontal axis indicates a time and a vertical axis indicates an exercise intensity and a heart rate. FIG. 11A indicates an exercise intensity (thin continuous line) and an actual heart rate measured by the heart rate sensor 16 (thick continuous line). FIG. 11B indicates an exercise intensity (thin continuous line), the result of estimating a heart rate according to the related art (broken line), and the result of estimating a heart rate according to the present technique (thick continuous line).

In sections A, C, and E where an exercise intensity is low, the user stops or hardly exercises. In a section D where an exercise intensity is high, the user actively exercises (e.g., running). Furthermore, in sections B and F, each being located between the range of a high exercise intensity and the range of a low exercise intensity, the user moderately exercises (e.g., walking).

As indicated in FIG. 11A, a heart rate gradually increases if the user starts exercising from rest. The heart rate gradually decreases if the user stops exercising or shifts to a moderate exercise.

However, as indicated by a broken line in FIG. 11B, the technique of estimating a heart rate according to the related art can estimate only rapid fluctuations synchronized with an exercise intensity, leading to a result deviating from an actual heart rate.

In contrast, as indicated by the thick continuous line in FIG. 11B, the result of estimating a heart rate according to the present technique does not rapidly fluctuate even when an exercises intensity changes, thereby estimating a heart rate close to a measured value of the heart rate sensor 16, the measured value gradually increasing and decreasing.

According to the present technique, the user only needs to wear the acceleration sensor or a device having the function of the acceleration sensor in order to estimate biological information. This hardly causes inconvenience or a burden on the user and achieves inexpensive estimation of a heart rate with ease.

According to the present technique, a heart rate can be estimated even if a heart rate of the user wearing the heart rate sensor cannot be accurately measured or the user does not wear the heart rate sensor. A heart rate cannot be measured in the following case: the user wearing the heart rate sensor is actively exercising during sports and thus the heart rate cannot be correctly measured. Alternatively, a heart rate may be incorrectly measured if the user wearing the heart rate sensor suffers from vasoconstriction because of environmental factors such as a low temperature. Moreover, the user may be unable to wear the heart rate sensor because of a dress code or inconvenience during an exercise. Furthermore, the heart rate sensor may be unusable in view of cost and power saving.

### 2. Application Example

An application example of the present technique will be described below.

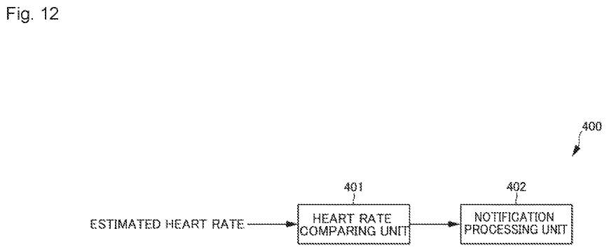

First, in a first application example, an estimated heart rate is supplied from the second biological information processor 200 to a notification device 400 illustrated in FIG. 12. The notification device 400 includes a heart rate comparing unit 401 that compares an estimated heart rate and a predetermined threshold value and a notification processing unit 402, and is configured to issue an alert to notify the user that an estimated heart rate is higher than the threshold value.

The notification device 400 may be provided in the device 10 where the second biological information processor 200 operates, may operate in a device different from the device 10, or may be a separate device.

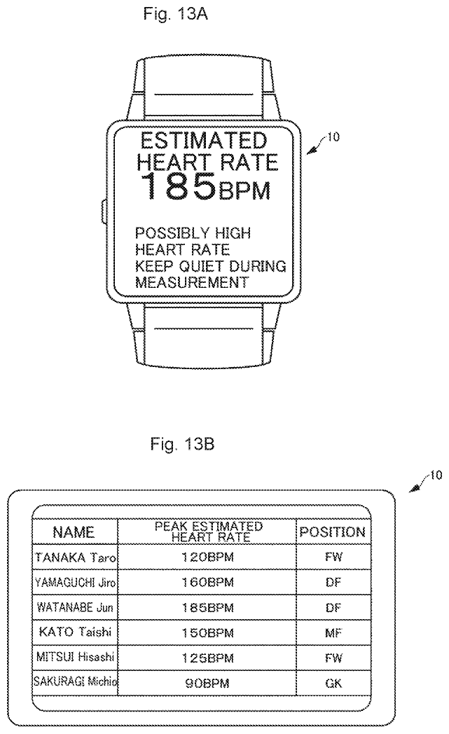

A notification can be provided by, for example, an audio output function, a vibrating function, and a display function on a display. These functions are typically provided for the device 10 where the second biological information processor 200 operates. For example, if the device 10 is a wearable device as illustrated in FIG. 13A, an estimated heart rate and a message sent to the user are displayed on the display.

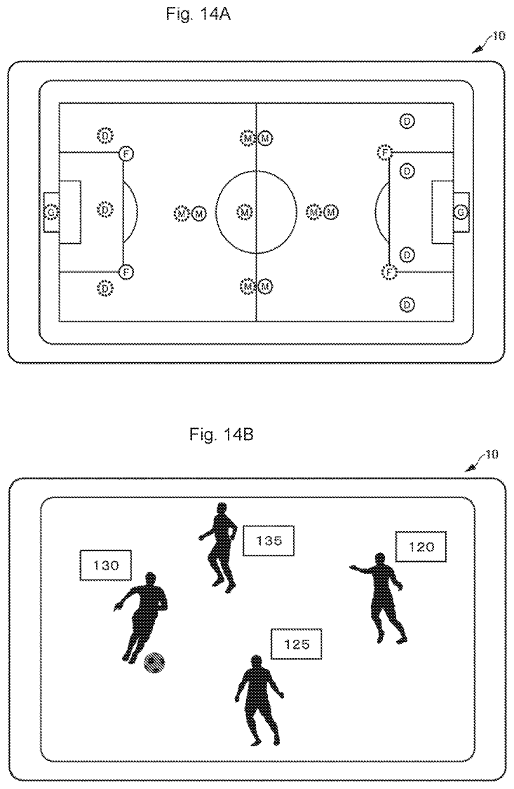

If the device 10 is a tablet with a large display as illustrated in FIG. 13B, estimated heart rates may be simultaneously displayed for multiple users. As illustrated in FIGS. 14A and 14B, estimated heart rates may be displayed at the positions of users tracked by cameras during a game or practice of sports such as soccer. The estimated heart rates may be displayed as a color map as well as numeric values.

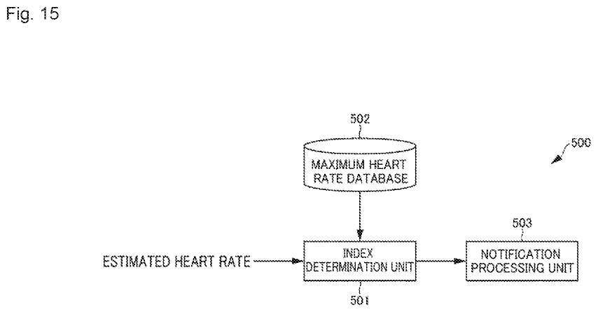

In a second application example, an estimated heart rate is supplied from the second biological information processor 200 to a determination device 500 illustrated in FIG. 15. The determination device 500 includes an index determination unit 501 that determines a predetermined index on the basis of an estimated heart rate and a maximum heart rate, a maximum heart rate database 502, and a notification processing unit 503. The maximum heart rate can be a value obtained from the age of the user (220—age) or can be estimated from a heart rate during a known exercise. Thus, the user needs to register the maximum heart rate in advance in the maximum heart rate database 502.

In response to the determination result of the index determination unit 501, the notification processing unit 503 displays index information in, for example, the device 10 to notify the user of the information. The predetermined index is, for example, FatBurnZone that is a heart rate zone where fat can be efficiently burned, a training effect index that is a heart rate zone where the effect of training is maximized, or a degree of fatigue.

The determination device 500 may be provided in the device 10 where the second biological information processor 200 operates, may operate in a device different from the device 10, or may be a separate device.

In a third application example, a heart rate measured by the heart rate sensor 16 and a heart rate estimated by the second biological information processor 200 are separately displayed at the same time in, for example, the device 10 as a log of heart rates.

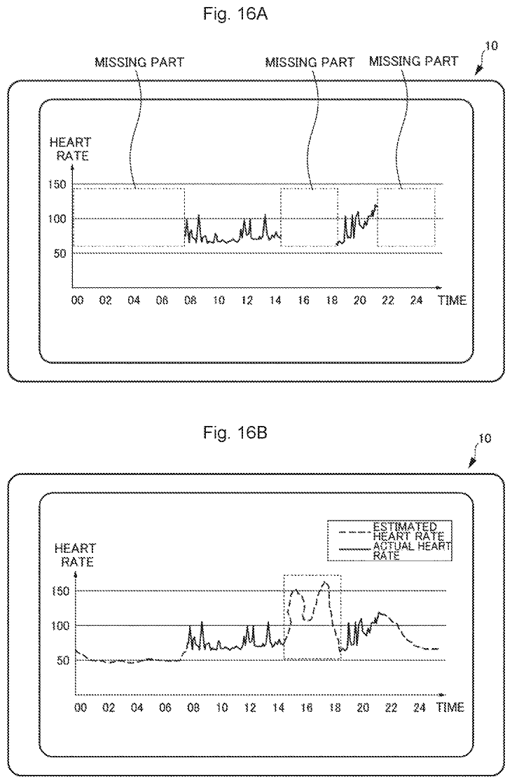

For example, it is assumed that the user who measures a heart rate every day with a wearable device having the function of a heart rate sensor and records the heart rate as a log forgets to wear the wearable device and has a missing log of a heart rate. Conventionally, the missing log of a heart rate is displayed as indicated in FIG. 16A.

In the present technique, however, even if the heart rate sensor 16 is not provided, a heart rate can be estimated by using an acceleration measured by another device (e.g., a smartphone) having an acceleration sensor or the function of an acceleration sensor in a period during which an actual heart rate cannot be measured. Thus, as indicated in FIG. 16B, an actual heart rate and an estimated heart rate can be displayed as a log on the device 10 or the like. In this case, the actual heart rate and the estimated heart rate are preferably displayed to be discriminated from each other. This can prevent the user who follows a daily habit of measuring a heart rate from lacking motivation when the user cannot measure a heart rate in a certain period.

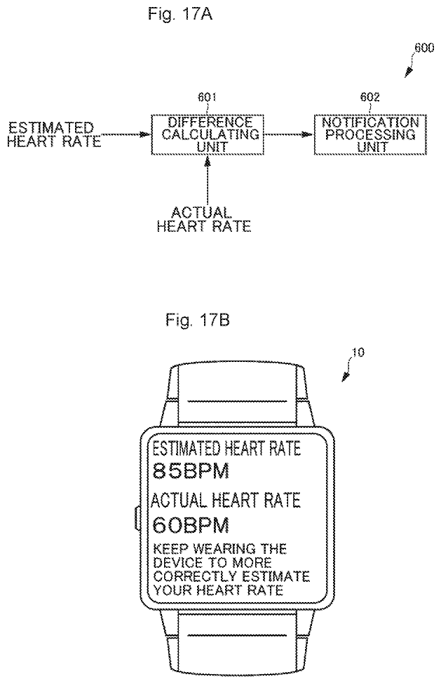

In a fourth application example, a difference calculator 600 in FIG. 17A receives an estimated heart rate from the second biological information processor 200 and an actual heart rate from the heart rate sensor 16. The difference calculator 600 includes a difference calculating unit 601 that calculates a difference between an actual heart rate and an estimated heart rate and a notification processing unit 602, and is configured to provide a notification for the user when a difference is equal to or larger than a predetermined value.

In the case of a large difference between an actual heart rate and an estimated heart rate, the correlation database 300 is insufficiently constructed, that is, it is necessary to store more acceleration feature amounts AC_{feat} and convergence heart rates HR^{(x)}ₚₗₐₜ in the correlation database 300.

Thus, if an actual heart rate of the user can be acquired from the heart rate sensor 16 even after the correlation database 300 is constructed, whether the correlation database 300 is sufficiently constructed can be determined from a difference between an actual heart rate and an estimated heart rate, and the user can be notified of the result.

If the absolute value of a difference between an actual heart rate and an estimated heart rate is equal to or higher than a predetermined threshold value (e.g., 20 BPM (Beat Per Minute) on average per day), a notification is provided to encourage the user to wear the heart rate sensor 16 and construct the correlation database 300 as illustrated in FIG. 17B on the assumption that the correlation database 300 is insufficiently constructed.

The difference calculator 600 may be provided in the device 10 where the second biological information processor 200 operates, may operate in a device different from the device 10, or may be a separate device.

In a fifth application example, an estimated heart rate is used when a heart rate is calculated from the frequency of a blood density change (PPG(Photoplethysmography) signal).

The waveform of the PPG signal is similar to that of an arterial blood pressure, so that a heart rate can be used for calculation. However, the PPG signal is acquired by measuring irradiating a skin with LED light and measuring a change of the intensity of reflected light in a bloodstream. Thus, in the event of extraneous disturbance caused by reflected waves or body motions, a peak not caused by a heart rate or a heart beat may be erroneously detected and result in heart beat calculation with lower accuracy.

Thus, a predetermined tolerance is set for an estimated heart rate and is outputted as an estimated heart rate±tolerance. The tolerance is changed according to the degree of maturity in the estimation of a heart rate. Furthermore, a heart rate corresponding to a frequency bin having peak power in the estimated heart rate±tolerance from the spectrogram of the PPG signal is set as an output heart rate.

In a sixth application example, a heart rate is estimated by using video captured by a camera instead of an acceleration measured by the acceleration sensor 17. This application example is useful, for example, in the case where the user can wear the heart rate sensor 16 and the acceleration sensor 17 in a period during which the correlation database is constructed in practice, but the user cannot wear the heart rate sensor 16 or the acceleration sensor 17 when a heart rate is estimated in a game or performance.

Since the user can wear the heart rate sensor 16 and the acceleration sensor 17 in a period during which the correlation database is constructed in practice, the first biological information processor 100 is identical to that of the embodiment.

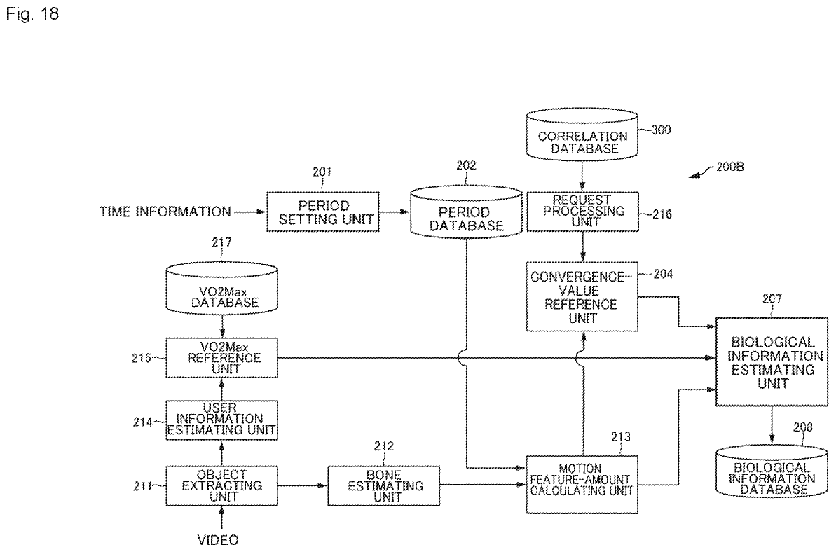

FIG. 18 illustrates the configuration of a second biological information processor 200B according to the sixth application example. The second biological information processor 200B includes an object extracting unit 211, a bone estimating unit 212, a motion feature-amount calculating unit 213, a user information estimating unit 214, a VO2Max reference unit 215, a request processing unit 216, and a VO2Max database 217 in addition to the configuration illustrated in FIG. 5. MOTION_{feat} and the convergence heart rate HR^{(x)}ₚₗₐₜ are stored in advance in the correlation database 300 so as to be associated with each other.

The object extracting unit 211 extracts a scene including a target user whose heart rate is to be estimated, from video captured by the camera according to a known subject recognition technique or face recognition technique.

The bone estimating unit 212 estimates a bone of the user from the extracted scene by analysis using a DNN (Deep Neural Network).

The motion feature-amount calculating unit 213 estimates the travel distance or the traveling speed of a bone gravity from the result of bone estimation and estimates a momentum from the angular speed of a leg part with respect to the lower back. The estimated information is denoted as MOTION_{feat}.

By transmitting a request to the request processing unit 216, the convergence-value reference unit 204 acquires the convergence heart rate HR^{(x)}ₚₗₐₜ corresponding to MOTION_{feat} from the correlation database 300.

The user information estimating unit 214 estimates user information including the age and sex of the user by, for example, analysis using the DNN (Deep Neural Network) on the basis of target user information from the object extracting unit 211.

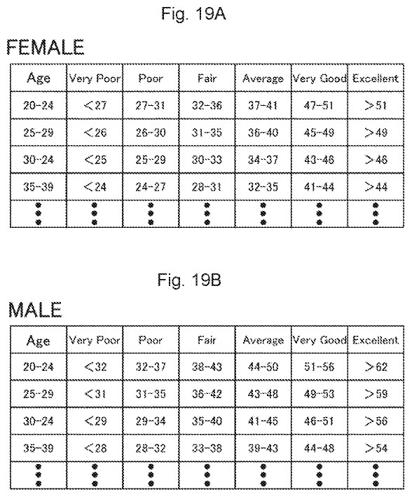

The VO2Max reference unit 215 acquires, based on the user information, VO2Max with reference to the VO2Max database 217 in which ages and sexes are associated with VO2Max in advance for males and females as indicated in FIGS. 19A and 19B. The degrees of VO2Max (e.g., Poor, Good in FIG. 19) relative to the ages and sexes of users of the VO2Max database 217 are associated by estimating weights and body sizes.

The biological information estimating unit 207 then calculates a heart rate HR(t) at any time t on the basis of VO2Max, MOTION_{feat}, and the convergence heart rate HR^{(x)}ₚₗₐₜ.

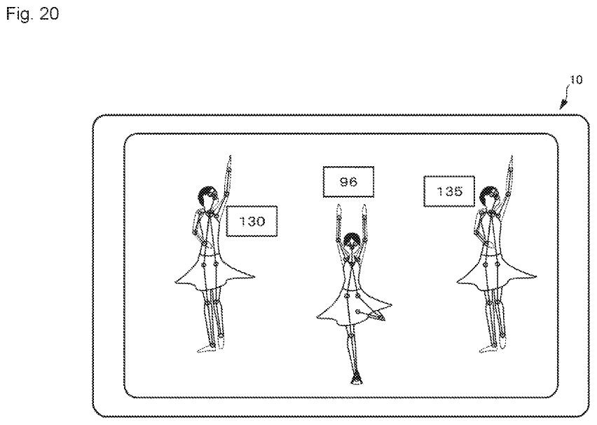

In the sixth application example, the user does not need to wear the acceleration sensor 17 to estimate a heart rate. Thus, for example, this application example is useful for estimating a heart rate of the user who cannot wear the acceleration sensor 17 or the device 10 as in a ballet illustrated in FIG. 20. As illustrated in FIG. 20, bone estimation is performed on users acting as subjects during performance, and estimated heart rates can be simultaneously superimposed and displayed.

In a seventh application example, a heart rate is estimated by using sensor information from a remote heart rate sensor installed at a limited position. For some remote heart rate sensors, for example, the microwave Doppler effect is used. The installation is limited because of the cost and the absence of the installation position (outdoor). A marathon race, in which a remote heart rate sensor can be installed in a facility, e.g., a stadium but cannot be installed in an urban area, will be described below as an example.

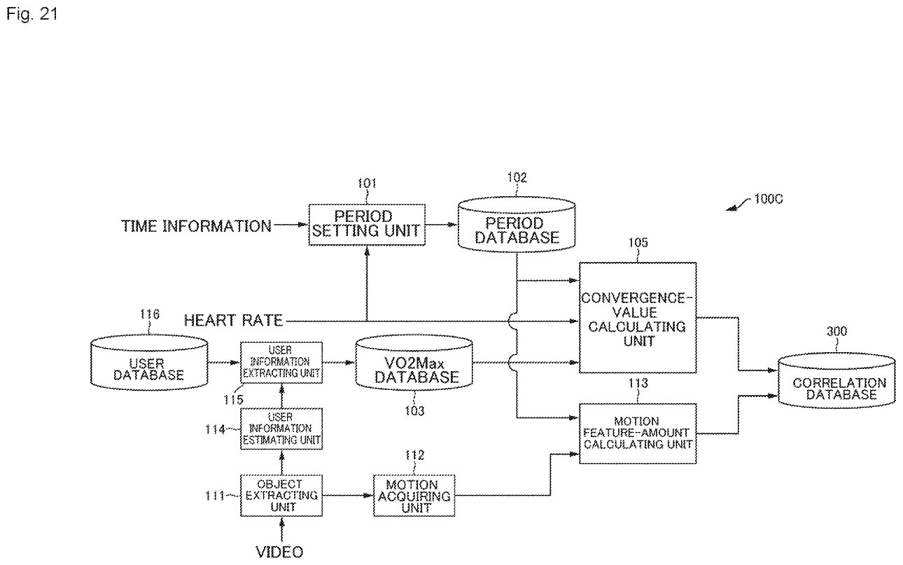

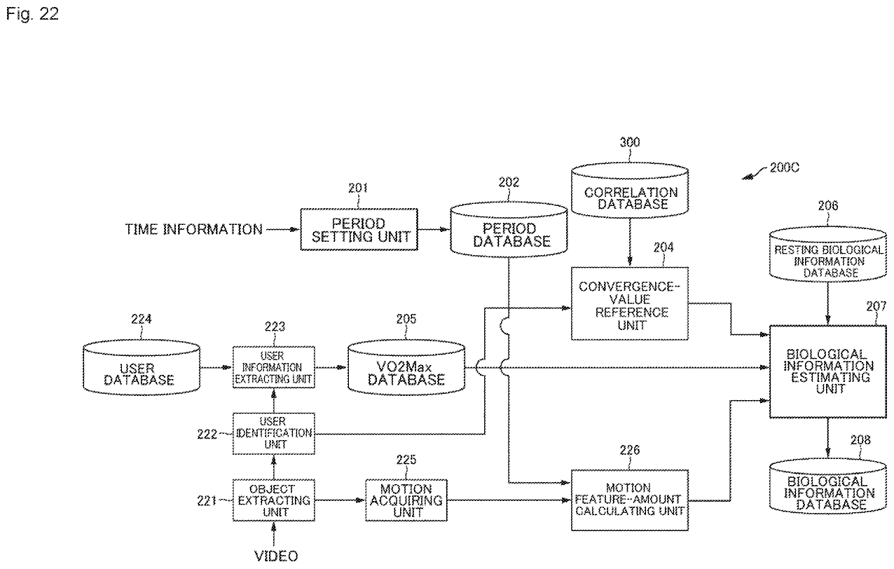

A first biological information processor 100C according to the seventh application example can be configured as illustrated in FIG. 21. A second biological information processor 200C can be configured as illustrated in FIG. 22.

In a stadium where a remote heart rate sensor can be installed, a heart rate of the user is measured by the remote heart rate sensor and a motion feature amount MOTION_{feat} is measured from video captured by a camera. The motion feature amount MOTION_{feat} is measured from the video of the camera as in the sixth application example. The correlation database 300 can be constructed by a first biological information processor 100B in a stadium that is an installation section of the remote heart rate sensor and the starting point of the marathon race.

In other sections such as an urban area where the remote heart rate sensor can be installed, a heart rate of a marathon runner can be estimated by the second biological information processor 200C by using video captured by the camera and the correlation database 300. In the seventh application example, a marathon runner as a subject does not need to wear the acceleration sensor 17 or the device 10. This prevents inconvenience to the marathon runner or an adverse effect on running.

In order to match an estimation target user to be detected in the installation section of the remote heart rate sensor and other sections, a user ID determination unit needs to identify a user as a target of the construction of the correlation database 300 by face recognition via the DNN of the camera and use the correlation database 300, which is constructed in the installation section of the remote heart rate sensor, to estimate a heart rate of the user having the same user ID in other sections.

In an eighth application example, a heart rate of the user is measured by a remote heart rate sensor installed in the house of the user to construct the correlation database 300, and the user wears only the acceleration sensor 17 away from home while a heart rate of the user is estimated. The user does not always need to wear the heart rate sensor 16 and the acceleration sensor 17. An estimated heart rate can be obtained by wearing the acceleration sensor 17 alone.

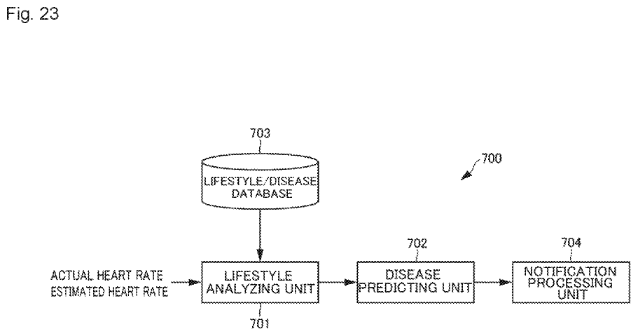

The result of heart rate estimation can be used for analyzing the lifestyle of the user. For example, as illustrated in FIG. 23, an estimated heart rate is supplied to a lifestyle analyzer 700. The lifestyle analyzer 700 includes a lifestyle analyzing unit 701, a disease predicting unit 702, a lifestyle/disease database 703, and a notification processing unit 704.

The lifestyle analyzing unit 701 receives, as inputs, an actual heart rate from the heart rate sensor 16 and an estimated heart rate from the second biological information processor 200, calculates an exercise intensity, a frequency, and a duration or the like, and analyzes the lifestyle of the user. The disease predicting unit 702 obtains a disease prediction result with reference to the analysis result and the lifestyle/disease database 703 that is a collection of probability medical findings about diseases such as diabetes in a lifestyle. The notification processing unit 704 notifies the user of the disease prediction result by displaying the result on the device 10.

In a ninth application example, a blood sugar level is continuously estimated in a noninvasive manner by setting, as a blood sugar level, biological information on the user serving as a subject, setting insulin secretory ability as a parameter, and setting food intake as a user exercise.

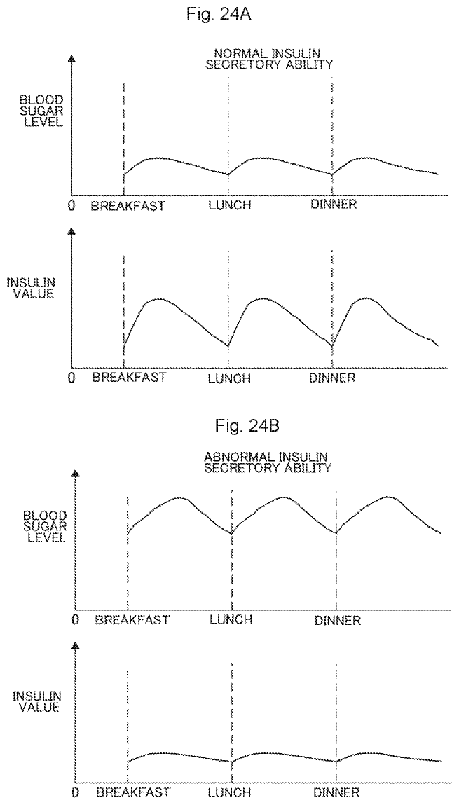

The insulin secretory ability is acquired in advance as Insulinogenic Index by an oral glucose tolerance test of 75 g. Moreover, the contents of meals are recorded, a reduction in meals (food intake) is measured by a mass sensor, a mealtime is measured by a clock, and the information is collected in the device 10 for estimating a blood sugar level. As indicated in FIG. 24, time series variations in blood sugar level are different for the same contents of meals because of a difference in insulin secretory ability. Thus, by using insulin secretory ability as a parameter, a blood sugar level can be estimated with higher accuracy. For example, the application example can be used to contribute to the health management of the user such that an estimated blood sugar level is compared with a threshold value and if the estimated blood sugar level is equal to or higher than the threshold value, the user is notified of the result.

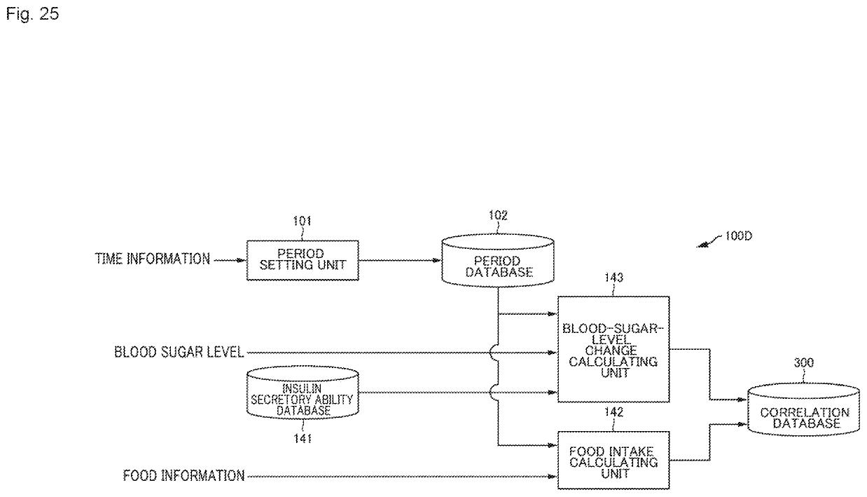

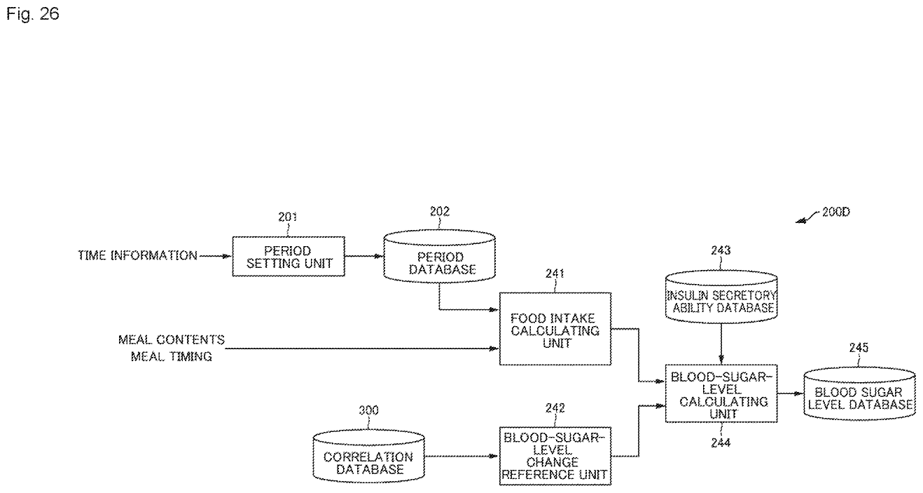

In the ninth application example, a first biological information processor 100D and a second biological information processor 200D are configured as illustrated in FIGS. 25 and 26.

In a tenth application example, a blood alcohol concentration is continuously estimated by setting, as a blood alcohol concentration, biological information on the user serving as a subject, setting alcohol metabolizing ability as a parameter, and setting driving as a user exercise.

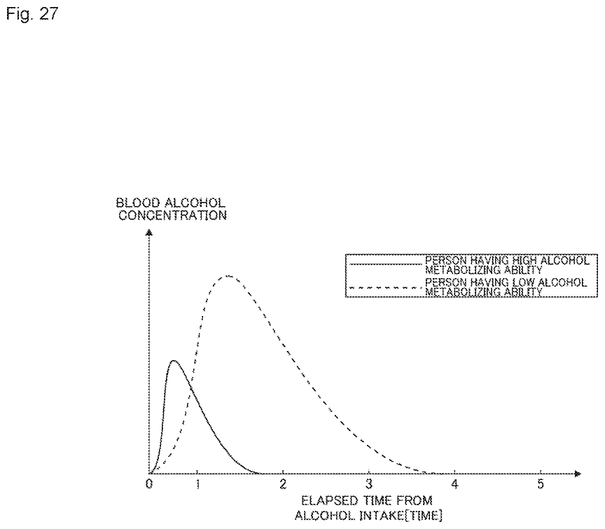

The alcohol metabolizing ability is acquired by a blood test or a patch test. As indicated in FIG. 27, time series variations are different for the same alcohol intake because of a difference in alcohol metabolizing ability. Thus, a blood alcohol concentration can be estimated with higher accuracy by using alcohol metabolizing ability as a parameter. For example, the application example can be used to contribute to the health management of the user such that an estimated blood alcohol concentration is compared with a maximum threshold value (e.g., 0.08%) and a minimum threshold value (e.g., 0.05%), a notification to keep drinking to a minimum is provided for the user if the estimated blood alcohol concentration is equal to or higher than the maximum threshold, and a notification to permit drinking is provided for the user if the estimated blood alcohol concentration is equal to or lower than the minimum threshold value.

In an eleventh application example, an electrocardiogram or a pulse waveform is used as biological information, and a biological characteristic parameter such as an arteriosclerosis level or an artery diameter is used as a first parameter in order to estimate a systolic blood pressure and a diastolic pressure.

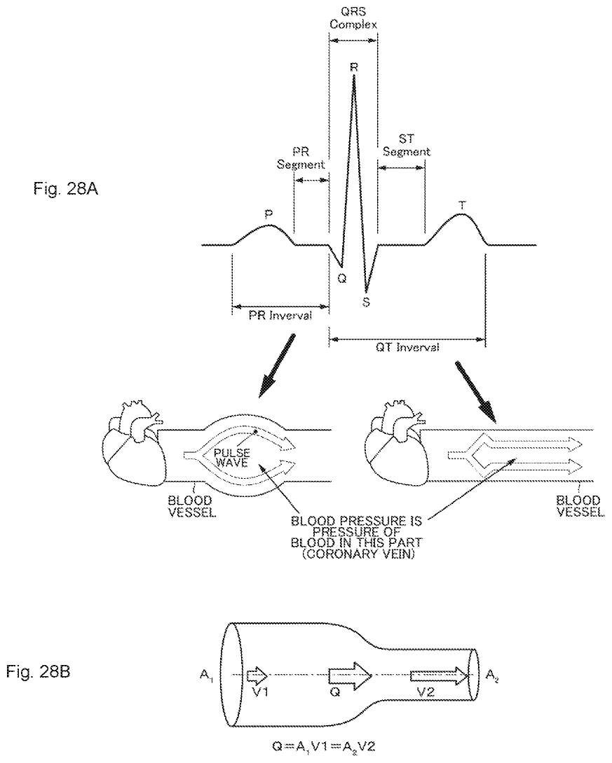

As indicated in FIG. 28A, a blood volume ejected from a heart with respect to time can be recognized on the basis of the waveform of an electrocardiogram. To recognize the blood volume, it is necessary to measure the electrocardiogram in advance and measure a blood flow rate in a coronary artery.

Moreover, it is found that a low arteriosclerosis level (flexible artery) expands a blood vessel in response to a pulsation of blood and thus does not cause a higher blood pressure than a high arteriosclerosis level (hard artery).

As indicated in FIG. 28B, it is found that a coronary artery having a large diameter has a low pressure with respect to a blood volume ejected from the same heart according to the continuity equation of a normal incompressible fluid.

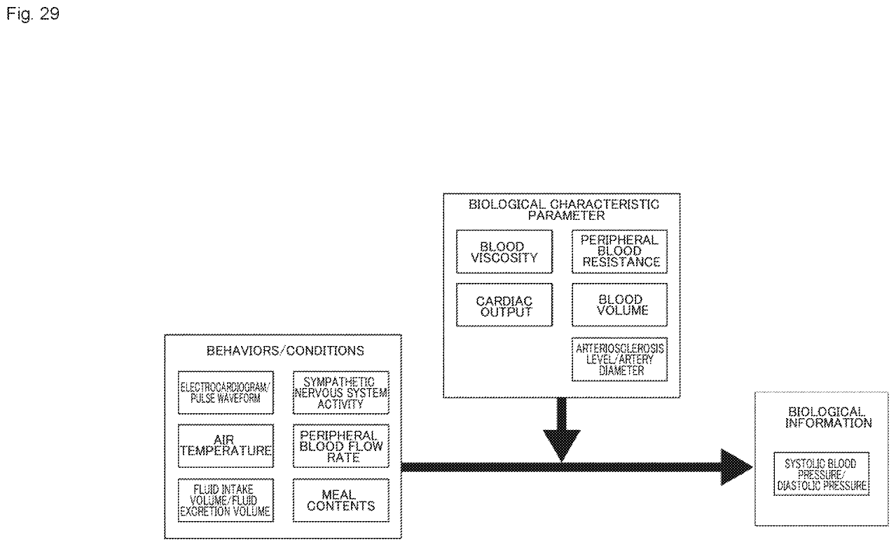

FIG. 29 illustrates an example of behaviors/conditions and biological characteristic parameters that can be used for estimating a systolic blood pressure and a diastolic pressure. The behaviors/conditions and biological parameters are preferably used in combinations rather than alone.

By using information about behaviors/conditions is used as biological information and using the biological characteristic parameter as a first parameter, a systolic blood pressure and a diastolic pressure can be estimated from the correlation relationship according to the present technique.

### 3. Modification Example

The embodiment of the present technique has been described specifically, but the present technique is not limited to the above-described embodiment and various modifications can be made based on the technical sprit of the present technique.

The present technique can be also configured as follows:

(1) A biological information processor including a biological information estimating unit that estimates biological information on a subject in a second period on the basis of a correlation between a first parameter in a first period and biological information in the first period and the first parameter in the second period subsequent to the first period, the first parameter being acquired in advance for the subject.
(2) The biological information processor according to (1), wherein the biological information is a heart rate.
(3) The biological information processor according to (2), wherein the biological information in the first period is a heart rate that converges when an exercise by the subject continues in the first period.
(4) The biological information processor according to (3), wherein the heart rate that converges when the exercise by the subject continues in the first period is calculated on the basis of the biological information at the start and end of the first period and a second parameter.
(5) The biological information processor according to (4), wherein the second parameter is cardiorespiratory endurance (VO2Max).
(6) The biological information processor according to any one of (1) to (5), wherein the biological information estimating unit estimates the biological information at any time in the second period on the basis of the biological information that converges when the exercise by the subject continues in the second period.
(7) The biological information processor according to (6), wherein the biological information estimating unit estimates the biological information at any time in the second period on the basis of the second parameter, the biological information at rest, and the duration of the exercise of the subject.
(8) The biological information processor according to (5), wherein the second parameter is used after being multiplied by a coefficient based on the relationship between the second parameter and the biological information when a constant exercise intensity is continued.
(9) The biological information processor according to any one of (1) to (8), wherein the first parameter is a value calculated from the acceleration of the exercise of the subject.
(10) The biological information processor according to any one of (1) to (9), wherein the correlation between the first parameter in the first period and the biological information in the first period is stored in a correlation database before the second period.
(11) The biological information processor according to (10), wherein the biological information in the first period is acquired with reference to the correlation database on the basis of the first parameter.
(12) The biological information processor according to any one of (1) to (11), wherein the second period is a period during which the biological information is unobtainable, a period during which the biological information is not correctly obtainable, or a period during which the biological information is obtainable in a limited way, and is a period during which the first parameter is obtainable.
(13) The biological information processor according to any one of (1) to (12), wherein the first parameter is acquired from acceleration information measured by an acceleration sensor.
(14) The biological information processor according to any one of (1) to (13), wherein the biological information in the first period is acquired from video captured by a camera.
(15) The biological information processor according to any one of (1) to (14), wherein the biological information is a blood sugar level.
(16) The biological information processor according to any one of (1) to (14), wherein the biological information is a blood alcohol concentration.
(17) The biological information processor according to any one of (1) to (16), wherein the biological information is an electrocardiogram or a pulse waveform.
(18) A biological information processing method includes estimating biological information on a subject in a second period on the basis of a correlation between a first parameter in a first period and biological information in the first period and the first parameter in the second period subsequent to the first period, the first parameter being acquired in advance for the subject.
(19) A biological information processing program that causes a computer to perform a biological information processing method including estimating biological information on a subject in a second period on the basis of a correlation between a first parameter in a first period and biological information in the first period and the first parameter in the second period subsequent to the first period, the first parameter being acquired in advance for the subject.

### REFERENCE SIGNS LIST

100 First biological information processor
200 Second biological information processor
207 Biological information estimating unit

1. A biological information processor comprising:
a biological information estimating unit that estimates biological information on a subject in a second period on a basis of a correlation between a first parameter in a first period and biological information in the first period and the first parameter in the second period subsequent to the first period, the first parameter being acquired in advance for the subject.

2. The biological information processor according to claim 1, wherein
the biological information is a heart rate.

3. The biological information processor according to claim 2, wherein
the biological information in the first period is a heart rate that converges when an exercise by the subject continues in the first period.

4. The biological information processor according to claim 3, wherein
the heart rate that converges when the exercise by the subject continues in the first period is calculated on a basis of the biological information at start and end of the first period and a second parameter.

5. The biological information processor according to claim 4, wherein
the second parameter is cardiorespiratory endurance (VO2Max).

6. The biological information processor according to claim 1, wherein
the biological information estimating unit estimates the biological information at any time in the second period on a basis of the biological information that converges when the exercise by the subject continues in the second period.

7. The biological information processor according to claim 6, wherein
the biological information estimating unit estimates the biological information at any time in the second period on a basis of the second parameter, the biological information at rest, and a duration of the exercise of the subject.

8. The biological information processor according to claim 5, wherein
the second parameter is used after being multiplied by a coefficient based on a relationship between the second parameter and the biological information when a constant exercise intensity is continued.

9. The biological information processor according to claim 1, wherein
the first parameter is a value calculated from an acceleration of the exercise of the subject.

10. The biological information processor according to claim 1, wherein
the correlation between the first parameter in the first period and the biological information in the first period is stored in a correlation database before the second period.

11. The biological information processor according to claim 10, wherein
the biological information in the first period is acquired with reference to the correlation database on a basis of the first parameter.

12. The biological information processor according to claim 1, wherein
the second period is a period during which the biological information is unobtainable, a period during which the biological information is not correctly obtainable, or a period during which the biological information is obtainable in a limited way, and is a period during which the first parameter is obtainable.

13. The biological information processor according to claim 1, wherein
the first parameter is acquired from acceleration information measured by an acceleration sensor.

14. The biological information processor according to claim 1, wherein
the biological information in the first period is acquired from video captured by a camera.

15. The biological information processor according to claim 1, wherein
the biological information is a blood sugar level.

16. The biological information processor according to claim 1, wherein
the biological information is a blood alcohol concentration.

17. The biological information processor according to claim 1, wherein
the biological information is an electrocardiogram or a pulse waveform.

18. A biological information processing method comprising:
estimating biological information on a subject in a second period on a basis of a correlation between a first parameter in a first period and biological information in the first period and the first parameter in the second period subsequent to the first period, the first parameter being acquired in advance for the subject.

19. A biological information processing program that causes a computer to perform a biological information processing method comprising:
estimating biological information on a subject in a second period on a basis of a correlation between a first parameter in a first period and biological information in the first period and the first parameter in the second period subsequent to the first period, the first parameter being acquired in advance for the subject.
